(19) United States
(12) Patent Application Publication
(10) Pub. No.: US 2005/0119513 A1
(43) Pub. Date: Jun. 2, 2005

(54) Method for perfluoroalkylation by means of tris (perfluoroalkyl) phosphine oxides

(76) Inventors: Nikolai Ignatyev, Duisburg (DE); Urs Welz-Biermann, Heppenheim (DE); Michael Schmidt, Seeheim-Jugenheim (DE); Michael Weiden, Darmstadt (DE); Udo Heider, Winchester (DE); Helge Willner, Muehlheim-Ruhr (DE); Peter Sartori, Utting (DE); Alexej Miller, Duisburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/511,156

(22) PCT Filed: Mar. 17, 2003
(86) PCT No.: PCT/EP03/02741

(30) Foreign Application Priority Data
Apr. 16, 2002 (DE) 10216996.9

(51) Int. Cl. C07C 19/08

(52) U.S. Cl. 570/144; 570/171

(57) ABSTRACT

The invention relates to a method for perfluoroalkylation by means of tris(perfluoroalkyl)phosphine oxides.

The present invention relates to a process for perfluoroalkylation by means of tris(perfluoroalkyl)phosphine oxides.

Perfluoroalkylation is an important process for the preparation of fluorine-containing compounds, in particular organofluorine compounds. The perfluoroalkylating reagents usually employed are perfluoroalkyl halides, in particular perfluoroalkyl iodides, which function as a source of perfluoroalkyl groups (“Organofluorine Chemistry. Principles and Commercial Applications.” Edited by R. E. Banks, Plenum Press, New York 1994; G. G. Furin, “Some new aspects in the application of perfluoroalkyl halides in the synthesis of fluorine-containing organic compounds” (Review), Russ. Chem. Rev. (English Translation), 69, No. 6 (2000), pages 491-522; N. O. Brace, “Syntheses with perfluoroalkyl iodides. A review, Part III.”, J. of Fluorine Chem., 108 (2001), pages 147-175; N. O. Brace, “Syntheses with perfluoroalkyl iodides. Part II.”, J. of Fluorine Chem. 96 (1999), pages 101-127; N. O. Brace, “Syntheses with perfluoroalkyl radicals from perfluoroalkyl iodides. A rapid survey of synthetic possibilities with emphasis on practical applications. Part one: alkenes, alkynes and allylic compounds”, J. of Fluorine Chem., 96 (1999), pages 1-25; V. N. Boiko, “Ion-radical perfluoroalkylation. Part II.”, J. of Fluorine Chem., 69 (1994), pages 207-212).

In addition, perfluoroalkyl halides are employed for the preparation of organometallic compounds containing perfluoroalkyl, in particular trifluoromethyl, groups, which can themselves be employed for introducing perfluoroalkyl groups into organic molecules (D. J. Burton, “Fluorinated organometallics: perfluoroalkyl and functionalised perfluoroalkyl organometallic reagents in organic synthesis”, Tetrahedron, 48, No. 2 (1992), pages 189-275).

Furthermore, the reagent TMSCF₃ has been developed for nucleophilic trifluoromethylation (G. K. Surya Prakash, “Nucleophilic trifluoromethylation tamed”, J. of Fluorine Chem., 112 (2001), pages 123-131). This nucleophilic perfluoroalkylation process has been extended to further organic and inorganic substrates through the reaction of long-chain perfluoroalkyl iodides with tetrakis(dimethylamino)ethylene in the presence of chlorotrimethylsilane (V. A. Petrov, Tetrahedron Letters, 42 (2001), pages 3267-3269).

However, the above-mentioned perfluoroalkylation processes have the disadvantage that the corresponding perfluoroalkyl halides are either very expensive or their use is only allowed with very great restrictions in accordance with the Montreal Protocol, as, for example, in the case of the compound CF₃Br.

These disadvantages have resulted in the development of novel perfluoroalkylating reagents, as described in J. R. Desmurs et al., 12th European Symposium on Fluorine Chemistry, Berlin, Germany, 1998, Abstracts A23 and A24. However, these reagents can only be prepared using CF₃H, a highly volatile compound which is difficult to handle. Furthermore, other stable perfluoroalkylating reagents have been developed for nucleophilic trifluoromethylation, the synthesis of these reagents starting from the methyl hemiketal of fluoral, which first has to be prepared in a relatively complex process. In addition, the use of these reagents is restricted to trifluoromethylation (G. Blond et al., Tetrahedron Letters, 42 (2001), pages 2437-2475; T. Billard et al., Eur. J. Org. Chem., 2001, pages 1467-1471; T. Billard et al. Tetrahedron Letters, 41 (2000), pages 8777-8780; G. Blond et al., J. Org. Chem., 66, No. 14 (2001), pages 4826-4830).

The present invention therefore relates to the use of at least one tris(perfluoroalkyl)phosphine oxide for the perfluoroalkylation of chemical substrates.

Perfluoroalkylphosphine oxides are known. They can be prepared by reaction of difluorotris(perfluoroalkyl)phosphoranes with hexamethyldisiloxane [(CH₃)₃Si]₂O, as described in V. Ya. Semenii et al., Zh. Obshch. Khim., 55, No. 12 (1985), pages 2716-2720.

The fluoro(perfluoroalkyl)phosphoranes can be prepared by conventional methods known to the person skilled in the art.

These compounds are preferably prepared by electrochemical fluorination of suitable starting compounds, as described in V. Ya. Semenii et al., Zh. Obshch. Khim., 55, No. 12 (1985), pages 2716-2720; N. Igantiev et al., J. of Fluorine Chem., 103 (2000), pages 57-61 and WO 00/21969. The corresponding descriptions are incorporated herein by way of reference and are regarded as part of the disclosure.

The fluoro(perfluoroalkyl)phosphoranes employed as starting compounds can be prepared inexpensively by electrochemical fluorination.

For the perfluoroalkylation of chemical substrates using tris(perfluoroalkyl)phosphine oxides, it is necessary to treat the perfluoroalkyl-phosphine oxide with at least one base before or during the reaction with the substrate to be perfluoroalkylated. The perfluoroalkylation of the chemical substrate using at least one tris(perfluoroalkyl)phosphine oxide is preferably carried out in the presence of at least one base.

Preference is given here to strong bases, such as, for example, potassium tert-butoxide, n-butyllithium, metal amides and/or a Grignard reagent.

The perfluoroalkylation is preferably carried out in a suitable reaction medium, if necessary dried by conventional methods, such as, for example, cyclic or aliphatic ethers, in particular tetrahydrofuran or diethyl ether.

Suitable chemical substrates are preferably organic compounds, in particular tricoordinated organoboron compounds and organic compounds containing carbonyl groups.

The organoboron compounds employed are preferably tris(C₁₋₃)alkyl borates, particularly preferably trimethyl borate.

Preferred carbonyl group-containing compounds are optionally substituted diaryl ketone compounds, in particular benzophenone.

The perfluoroalkylation of chemical substrates using tris(perfluoroalkyl)-phosphine oxides can preferably be carried out under a water-free atmosphere, such as, for example, dry air, or an inert-gas atmosphere, such as, for example, argon or nitrogen.

The use of tris(perfluoroalkyl)phosphine oxides as perfluoroalkylating reagents is advantageous in particular since these compounds, in contrast to many other perfluoroalkylating reagents, are stable compounds, which enables them to be handled simply and safely.

The NMR spectra were recorded with the aid of a Bruker Avance 300 NMR spectrometer with the following frequencies:
300.1 MHz for ¹H
282.4 MHz for ¹⁹F and
96.3 MHz for ¹¹B.

The mass spectra were measured using an AMD 604 instrument.

The invention is explained below with reference to examples. These examples serve merely to explain the invention and do not restrict the general inventive idea.

### EXAMPLES

### Example 1

### Preparation of tris(pentafluoroethyl)phosphine Oxide

101.36 g (237.9 mmol) of difluorotris(pentafluoroethyl)phosphorane and 38.63 g (237.9 mmol) of hexamethyldisiloxane are refluxed with vigorous stirring for 1 hour in an FEP (fluoroethylene polymer) flask at a bath temperature of 30° C. until the gas formation of (CH₃)₃SiF subsides. The reaction mixture is subsequently heated at 110-120° C. (bath temperature) for 2 hours and distilled under atmospheric pressure. 86.5 g of tris(pentafluoroethyl)phosphine oxide, a clear and colourless liquid having a boiling point of 101° C., are obtained, corresponding to a yield of 90.0%, based on the difluorotris(pentafluoroethyl)phosphorane employed.

The resultant product is characterised by means of ¹⁹F- and ³¹P-NMR spectroscopy:
¹⁹F NMR spectrum; δ, ppm:
(Solvent CDCl₃, internal reference CCl₃F)
−79.3 t (CF₃); −117.3 dq (CF₂); J²_{P,F}=84.5 Hz; J³_{F,F}=2.5 Hz
³¹P NMR spectrum; δ, ppm:
(Solvent CDCl₃, reference 85% by weight H₃PO₄)
20.2 sep, J²_{P,F}=84.5 Hz

The values of the chemical shifts found correspond to the values disclosed in the publication by V. Ya. Seminii et al., Zh. Obshch. Khim., 55, No. 12 (1985), pages 2716-2720.

### Example 2

### Preparation of tris(n-nonafluorobutyl)phosphine Oxide

30.6 g (42.15 mmol) of difluorotris(n-nonafluorobutyl)phosphorane and 7.0 g (43.11 mmol) of hexamethyldisiloxane are refluxed with vigorous stirring for 5 hours in an FEP flask at a bath temperature of about 150-160° C. until the gas formation of (CH₃)₃SiF ceases. The reaction mixture is subsequently distilled under reduced pressure (1.6 kPa), and the fraction having a boiling point of 87-88° C. is collected. 26.1 g of the clear, colourless liquid of tris(n-nonafluorobutyl)phosphine oxide are obtained. The yield is 87.9%, based on the amount of difluorotris(n-nonafluorobutyl)-phosphorane employed.

The resultant product is characterised by means of ¹⁹F- and ³¹P-NMR spectroscopy:
¹⁹F NMR spectrum; δ, ppm:
(Solvent CDCl₃, internal reference ppm CCl₃F)
−81.2 t (CF₃); −112.5 dm (CF₂); −119.0 m (CF₂); −126.3 dm (CF₂); J⁴_{F,F}=9.5 Hz; J²_{P,F}=86.8 Hz
³¹P NMR spectrum; δ, ppm:
(Solvent CDCl₃, reference 85% by weight H₃PO₄)
24.20 sept.; J²_{P,F}=87.1 Hz

The values for the chemical shifts found correspond to the values disclosed in the publication by V. Ya. Seminii et al., Zh. Obshch. Khim., 55, No. 12 (1985), pages 2716-2720.

### Example 3

### Preparation of 2,2,3,3,3-pentafluoro-1,1-diphenylpropan-1-ol

a) 1.87 g (4.63 mmol) of tris(pentafluoroethyl)phosphine oxide are added slowly at −60° C. to a solution of 6 mmol of butyllithium (3 cm³ of a 2 M solution in cyclohexane) in 30 cm³ of dry tetrahydrofuran, during which the temperature should be kept below −55° C. The solution is stirred at this temperature for about 1 hour until the phosphine oxide has dissolved completely. A solution of 0.98 g (5.38 mmol) of benzophenone in 5 cm³ of dry tetrahydrofuran is subsequently added, and the mixture is warmed to room temperature over the course of 2 hours. The reaction mixture is treated with 20 cm³ of 0.1 N HCl and extracted with diethyl ether (2×50 cm³). The extract is washed with water (3×20 cm³) and dried over magnesium sulfate.

The ether is distilled off, and the desired product is crystallised from hexane, giving 0.42 g of 2,2,3,3,3-pentafluoro-1,1-diphenylpropan-1-ol, a white solid having a melting point of 82-83° C., corresponding to a yield of 30.0%, based on the phosphine, oxide employed.

The resultant product is characterised by means of ¹⁹F- and ¹H-NMR spectroscopy:
¹⁹F NMR spectrum; δ, ppm:
(Solvent CDCl₃, internal reference CCl₃F)
−77.6 s (CF₃); −116.9 m (CF₂)
¹H-NMR spectrum; δ, ppm:
(Solvent CDCl₃, reference TMS)
7.53-7.67 m (2H), 7.30-7.47 m (3H), 2.85 br.s (OH)

The values for the chemical shifts found and for the melting point correspond to the values disclosed in the publication by L. S. Chen et al., J. of Fluorine Chem., 20 (1982), pages 341—348.

b) 7 cm³ of a 2 M solution of butyllithium in cyclohexane are added to a solution of 1.98 g (12.27 mmol) of hexamethyldisilazane in 30 cm³ of dry tetrahydrofuran, and the mixture is heated for about 1 hour until the gas formation of butane is complete. 1.80 g (9.88 mmol) of benzophenone are added to the resultant solution of lithium bis(trimethylsilyl)amide, and the mixture is cooled to −60° C. 3.91 g (9.68 mmol) of tris(pentafluoroethyl)-phosphine oxide are added, during which the temperature should be kept below −55° C. The mixture is subsequently warmed to room temperature over the course of 2 hours. The reaction mixture is treated with 20 cm³ of 0.1 N HCl and extracted with diethyl ether (2×50 cm³). The extract is washed with water (3×20 cm³) and dried over magnesium sulfate. The ether is distilled off, and the desired product is crystallised from hexane, giving 0.70 g of 2,2,3,3,3-pentafluoro-1,1-diphenylpropan-1-ol, a white solid, corresponding to a yield of 23.9%, based on the phosphine oxide employed.

The melting point and NMR data correspond to the values indicated in Example 3a).

c) 2.00 g (4.95 mmol) of tris(pentafluoroethyl)phosphine oxide are added at −60° C. to 5.6 mmol of phenylmagnesium bromide in 40 cm³ of dry tetrahydrofuran, during which the temperature of the reaction mixture should be kept below −55° C. The reaction mixture is stirred at −45° C. for one hour, and 0.96 g (5.27 mmol) of benzophenone in 5 cm³ of dry tetrahydrofuran is added. The mixture is subsequently warmed to room temperature over the course of 2 hours. The reaction mixture is treated with 20 cm³ of 0.1 N HCl and extracted with diethyl ether (2×50 cm³). The extract is washed with water (3×20 cm³) and dried over magnesium sulfate.

The ether is distilled off, and the desired product is crystallised from hexane, giving 0.55 g of 2,2,3,3,3-pentafluoro-1,1-diphenylpropan-1-ol, a white solid, corresponding to a yield of 36.8%, based on the phosphine oxide employed.

The melting point and NMR data correspond to the values indicated in Example 3a).

### Example 4

### Potassium pentafluoroethyltrifluoroborate (C₂F₅)BF₃K

2.45 g (6.07 mmol) of tris(pentafluoroethyl)phosphine oxide were added at −40° C. to a mixture of 0.32 g (5.52 mmol) of spray-dried potassium fluoride and 1.72 g (16.55 mmol) of trimethyl borate (CH₃O)₃B in 3 cm³ of dry 1,2-dimethoxyethane, during which the temperature of the reaction mixture should be kept below −30° C. The reaction mixture was stirred at −30° C. for one hour and brought to room temperature. The solvent was distilled off, and the resultant residue was dissolved in 10 cm³ of diethyl ether. The solution was cooled using an ice bath, and 1.2 g of anhydrous hydrogen fluoride (HF) were added. The reaction mixture was stirred at room temperature for one hour, and the solvent was distilled off under an oil-pump vacuum. The residue was washed with chloroform (3×5 cm³) and dissolved in 10 cm³ of water. The aqueous phase was extracted with diethyl ether (5×10 cm³), and the aqueous solution was separated off. The water was distilled off at a pressure of 7 Pa, and the residue was dried at 40° C. in this vacuum for one hour, giving 0.67 g of potassium pentafluoroethyltrifluoroborate (C₂F₅)BF₃K in the form of a white solid. The yield was 53.6%, based on the potassium fluoride employed.

The resultant product was characterised by means of ¹¹B- and ¹⁹F-NMR spectroscopy:
¹¹B NMR spectrum; δ, ppm (solvent: acetonitrile-D₃; external reference BF₃ O(C₂F₅)₂): −0.2 tq, ¹J_{B,F}=41.0 Hz; ²J_{B,F}=20.0 Hz.
¹⁹F NMR spectrum; δ, ppm (solvent: acetonitrile-D₃; internal reference CCl₃F): −83.1 q, (CF₃); −135.9 q (CF₂); −152.9 q (BF₃); ¹J_{B,F}=41.1 Hz; ²J_{B,F}=19.6 Hz; ⁴J_{F,F}=5.0 Hz.

The corresponding signals correspond to the signals mentioned under Patent Application DE 102 16 998.5.

1. Process for perfluoroalkylation, characterised in that at least one tris-(perfluoroalkyl)phosphine oxide is reacted with the substrate to be perfluoroalkylated, where the tris(perfluoroalkyl)phosphine oxide is treated with at least one base before or during the reaction.

2. Process for perfluoroalkylation according to claim 1, characterised in that the substrates employed are organic compounds, preferably tricoordinated organoboron compounds and/or organic compounds containing carbonyl groups.

3. Process for perfluoroalkylation according to claim 1, characterised in that strong bases, preferably potassium tert-butoxide, n-butyllithium, metal amides and/or Grignard reagents, are used.

4. Use of at least one tris(perfluoroalkyl)phosphine oxide for the perfluoroalkylation of chemical substrates.
